(19) United States
(12) Patent Application Publication
(10) Pub. No.: US 2006/0100660 A1
(43) Pub. Date: May 11, 2006

(54) Blood clot filter configured for a wire guide

(75) Inventors: Thomas A. Osborne, Bloomington, IN (US); Mark R. Frye, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/268,355

(22) Filed: Nov. 7, 2005

Related U.S. Application Data
(60) Provisional application No. 60/625,900, filed on Nov. 8, 2004.

(51) Int. Cl. A61M 29/00

(52) U.S. Cl. 606/200

(57) ABSTRACT

A filter to capture blood clots includes a hub with a passageway through which a wire guide is received. The filter also includes a plurality of primary struts and a plurality of secondary struts that extend from the hub. Each primary strut terminates with a hook to anchor the filter in the blood vessel when the filter is deployed in the blood vessel. The secondary struts center the filter in the blood vessel as the secondary struts engage the interior of the blood vessel during deployment of the filter in the vessel.

### RELATED APPLICATION

This application claims the benefit of U.S. Provisional Application No. 60/625,900 filed Nov. 8, 2004, the entire contents of which are incorporated herein by reference.

### BACKGROUND

This invention relates to medical devices. More specifically, the invention relates to a removable vena cava clot filter.

Filtering devices that are percutaneously placed in the vena cava have been available for a number of years. A need for filtering devices arises in trauma patients, orthopedic surgery patients, neurosurgery patients, or in patients having medical conditions requiring bed rest or non-movement because of the likelihood of thrombosis in the peripheral vasculature of patients. The thrombi may break away from the vessel wall, and, depending on the size of the thrombi, pose a serious risk of pulmonary embolism when blood clots migrate from the peripheral vasculature through the heart and into the lungs.

A filtering device can be deployed in the vena cava of a patient when, for example, anticoagulant therapy is contraindicated or has failed. Typically, filtering devices are permanent implants even though the condition or medical problem that required the device has passed. Recently, filters have been employed or considered in preoperative patients and in patients predisposed to thrombosis, which, however, may increase the risk for pulmonary embolism in these patients.

Although the benefits of vena cava filters have been well established, improvements may be made. For example, filters generally have not been considered removable from a patient due to the likelihood of endotheliosis of the filter or fibrous reaction matter adherent to the endothelium during treatment. After deployment of a filter in a patient, proliferating intimal cells begin to accumulate around the filter struts that are in contact with the wall of the vessel. After a period of time, such ingrowth prevents removal of the filter without risk of trauma, requiring the filter to remain in the patient. As a result, there is a need for an effective filter that can be removed after the underlying medical condition has passed.

Although some filters have been designed to be removable from the vena cava, these filters commonly become off-centered or tilted with respect to the hub of the filter and the longitudinal axis of the vessel in which it has been inserted. As a result, these filters including the hub and the retrieval hook engage the vessel wall along their lengths and potentially become endothelialized within the vessel, making removal of the filters impossible or at least difficult.

### SUMMARY

In general, the present invention provides a filter that includes a hub and a plurality of primary struts and a plurality of secondary struts that extend from the hub. Each primary strut terminates with a hook to anchor the filter in the blood vessel when the filter is deployed in the blood vessel. The secondary struts center the filter in the blood vessel as the secondary struts engage the interior of the blood vessel during deployment of the filter.

To guide the filter through a vessel, the hub is provided with a passageway through which a wire guide is received. Thus, the wire guide can be extended through a sheath so that the terminal end of the wire guide can be placed near the site of interest. A medical specialist, such as a physician, can then push the filter along the wire guide to the desired location. Once the filter is deployed, both the sheath and wire guide are removed from the patient. The hub may be provided with a groove that engages with a retrieval device to remove the filter from the vessel.

Further features and advantages of this invention will become readily apparent from the following description, and from the claims.

### BRIEF DESCRIPTION OF THE DRAWINGS

FIG. 1 is an illustration of the anatomy the vena cava in which a filter is deployed in accordance with an embodiment of the invention.

FIG. 2 is a side perspective view of a vena cava filter in accordance with an embodiment of the invention.

FIG. 3 is a close-up view of a hub associated with filter shown in FIG. 2.

FIG. 4a is a cross-sectional view of the hub along the line 4-4 of FIG. 3.

FIG. 4b is a cross-sectional view of an alternative hub in accordance with the invention.

FIG. 5a is a cross-sectional view of a blood vessel showing the insertion of a wire guide.

FIG. 5b is a cross-sectional view of the blood vessel showing the insertion of a sheath and a vena cava filter over the wire guide.

FIG. 5c is a cross-sectional view of the blood vessel showing the vena cava filter partially deployed.

FIG. 6a is a cross-sectional view of the blood vessel showing the retraction of the sheath.

FIG. 6b is a cross-sectional view of the blood vessel showing the vena cava filter fully deployed.

FIG. 7 is a cross-sectional view of a blood vessel showing the vena cava filter of FIG. 2 deployed within the blood vessel.

FIG. 8 is a view of the blood vessel and filter of FIG. 7 taken along the line 8-8.

FIGS. 9a through 9e are interior views of the vena cava illustrating the removal of the vena cava filter.

### DETAILED DESCRIPTION

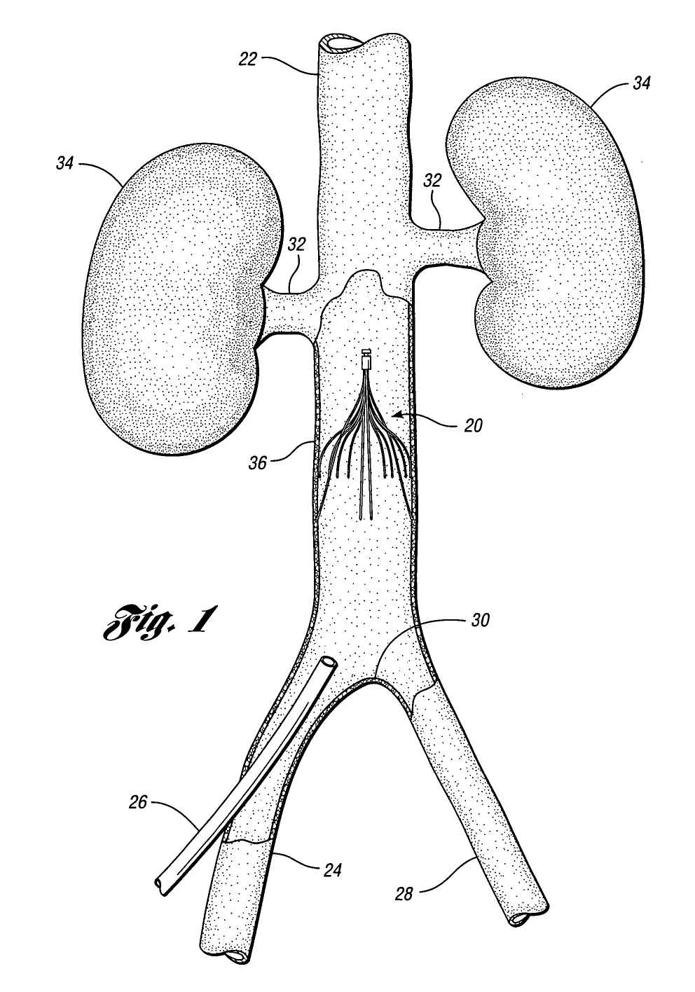

Turning now to the drawings, FIG. 1 illustrates a vena cava filter 20 embodying the principles of the present invention. The vena cava filter 20 is shown implanted in a vena cava 22 after it has been inserted through an iliac vein 24 with the use of a sheath 26. Alternatively, the vena cava filter 20 can be inserted through a jugular vein. As described below in greater detail, once implanted, the vena cava filter 20 is able to self align itself within the vena cava 22 to minimize endotheliosis of the filter. The vena cava filter 20 captures or lyses thrombi (or clots) carried through the vena cava 22 from the iliac veins 24, 28 toward the heart and into the pulmonary arteries, where clots can cause embolization. Moreover, the vena cava filter 20 is configured to minimize obstruction of flood flow through the vena cava 22.

The iliac veins 24, 28 from the legs merge into the vena cava 22 at a juncture 30, and the renal veins 32 from the kidneys 34 join the vena cava 22 downstream of the juncture 30. The portion of the vena cava between the juncture 30 and the renal veins 32 defines an inferior vena cava 36. In the illustrated embodiment, the length of a vena cava filter 20 is shorter than the length of the inferior vena cava 36. Otherwise, if the lower part of the filter 20 extends into the iliac veins 24, 28, the filtering effectiveness of the filter 20 may be compromised.

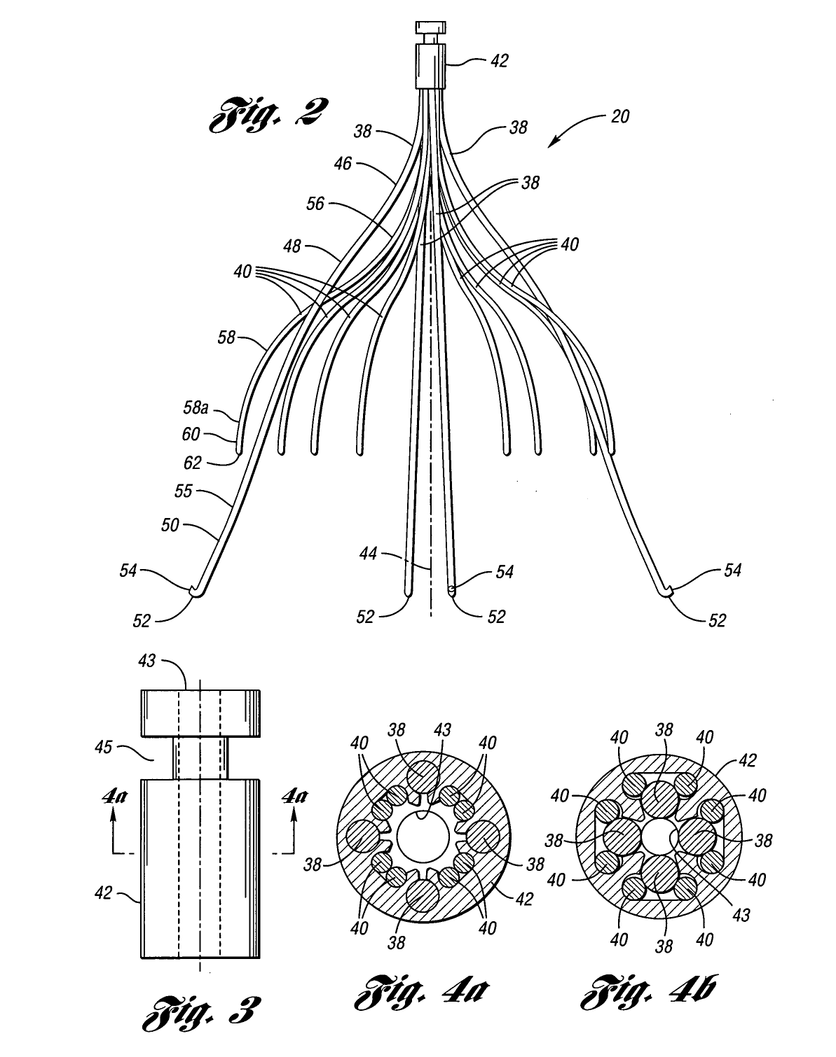

Referring now to FIGS. 2, 3, and 4, the filter 20 includes four primary struts 38 and eight secondary struts 40, each of which extends from a respective fixed end housed in a hub 42. To attach the fixed ends of the struts to the hub 42, the fixed ends are crimped together in a compact bundle about an opening or passageway 43, thereby defining a central or longitudinal axis 44. The diameter of this bundle is minimized to accommodate the size of the wires used to form the struts. The hub 42 is provided with a groove 45, which, as described below, engages with a retrieval device for removing the vena cava filter 20.

Each primary strut 38 is formed with a first curved section 46 that bends away form the central axis 44 and a second curved section 48 that bends away from the hub 42. A substantially straight section 50 extends from the second curved section 48 and terminates in an anchoring hook 52 with a barb 54. The section 50 may also have an additional curved section 55 that further flares the anchoring hooks 52 away from the central axis 44. Each primary strut 38 maintains a non-parallel relationship with the central axis 44 when the filter 20 is in its deployed configuration.

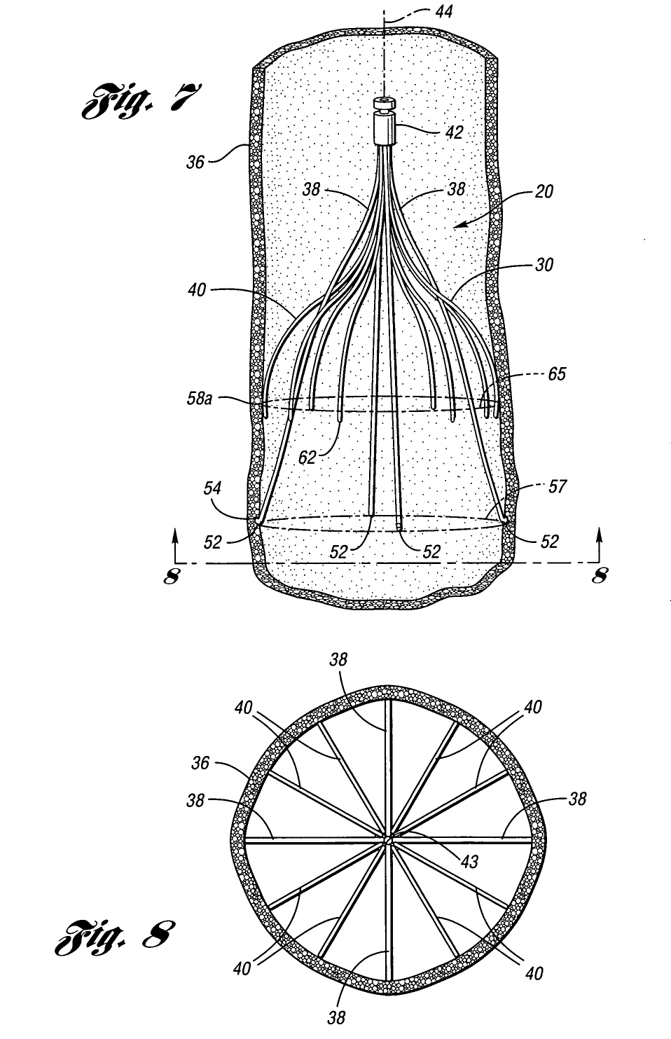

When the filter 20 is deployed in the blood vessel (see, for example, FIG. 7), the anchoring hooks 52 engage with the interior of the blood vessel in a first axial plane 57 aligned substantially perpendicular to the longitudinal axis of the blood vessel. The diameter of this plane of engagement 57 is about 30 mm or less.

The primary struts 38 have sufficient spring strength to move the hooks 52 to the interior wall, where the hooks 52, in particular, the barbs 54, anchor into the interior wall of the blood vessel to prevent the filter 20 from migrating from the delivery location of the filter in the blood vessel. In various embodiments, the primary struts 38 are formed from superelastic material, stainless steel wire, MP35N, Nitinol, elgiloy, chronichrome, cobalt chrome alloy or any other suitable material that will result in a self-opening or self-expanding filter. In certain embodiments, the primary struts 38 are formed from wire with a round or near round cross section with a diameter of at least about 0.015 inch. In other embodiments, the primary struts do not have a round cross-section. For example, the primary struts 38 can take on any shape with rounded edges to maintain non-turbulent blood flow. Rather than forming the struts from wire, they can be cut from a tube of any appropriate material by laser cutting, electrical discharge machining, or any other suitable process. Subsequently, the struts can be finished, for example, with an electropolishing process so that the resulting struts are substantially rounded.

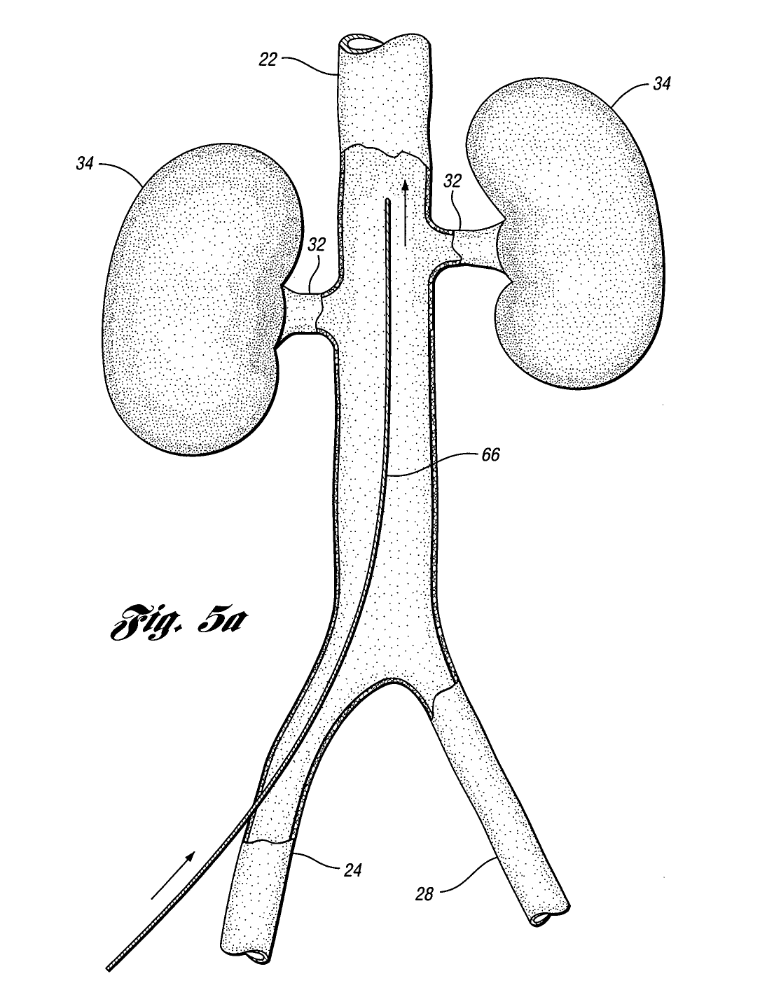

A pair of secondary struts 40 is positioned between adjacent primary struts 38 as shown in FIG. 4a, or, alternatively, a primary strut 38 is positioned between a pair of secondary struts 40 as shown in FIG. 4b. Each secondary strut 40 has a first curved section 56 that bends away from the central axis 44, a second curved or converging section 58 that bends toward the central axis 44, and an end section 60 that terminates in a tip 62 pointing toward the central axis 44. The tips 62 are located longitudinally between the hub 42 and the anchoring hooks 54 of the primary struts 38. To minimize the trauma to the vena cava caused by removing the filter 20, the free ends 60 of the secondary struts 40 do not have anchoring hooks.

When the filter 20 is in its deployed configuration, the outer regions 58a of the converging section 58 of each secondary strut 40 engage with the wall of the blood vessel. The radial force created between the secondary struts 40 and the wall of the blood vessel serves to align the filter 20 about the center of the blood vessel so that the central axis 44 is substantially parallel to the axis of the blood vessel.

When the filter 20 is deployed within the vessel, the outer regions 58a of the secondary struts 40 engage with the interior of the blood vessel in a second axial plane 65 (FIG. 7) that is substantially parallel to the first axial plane 57. The diameter of the second axial plane of engagement is also about 30 mm or less. As a result, the filter 20 has two layers or planes of struts longitudinally engaging the vessel wall. Note that the length of the primary struts 38 defines the length of the filter 20, since the secondary struts 40 do not extend further upstream than the primary struts 38. That is, the secondary struts 40 do not add to the overall length of the filter. In some embodiments, the length of the filter 20 is between about 3 cm and 7 cm. In a particular embodiment, the length of the filter is about 5 cm.

The secondary struts 40 can be made from the same type of material as the primary struts 38 and can be formed by the same process used to form the primary struts. However, the secondary struts may have round or near round cross section with a smaller diameter than the primary struts. In a particular embodiment, the diameter of the secondary struts is at least about 0.01 inch. The hub 42 can be made of any suitable material. For example, the hub 42 can be made from the same material as the primary struts and secondary struts to minimize the possibility of galvanic corrosion.

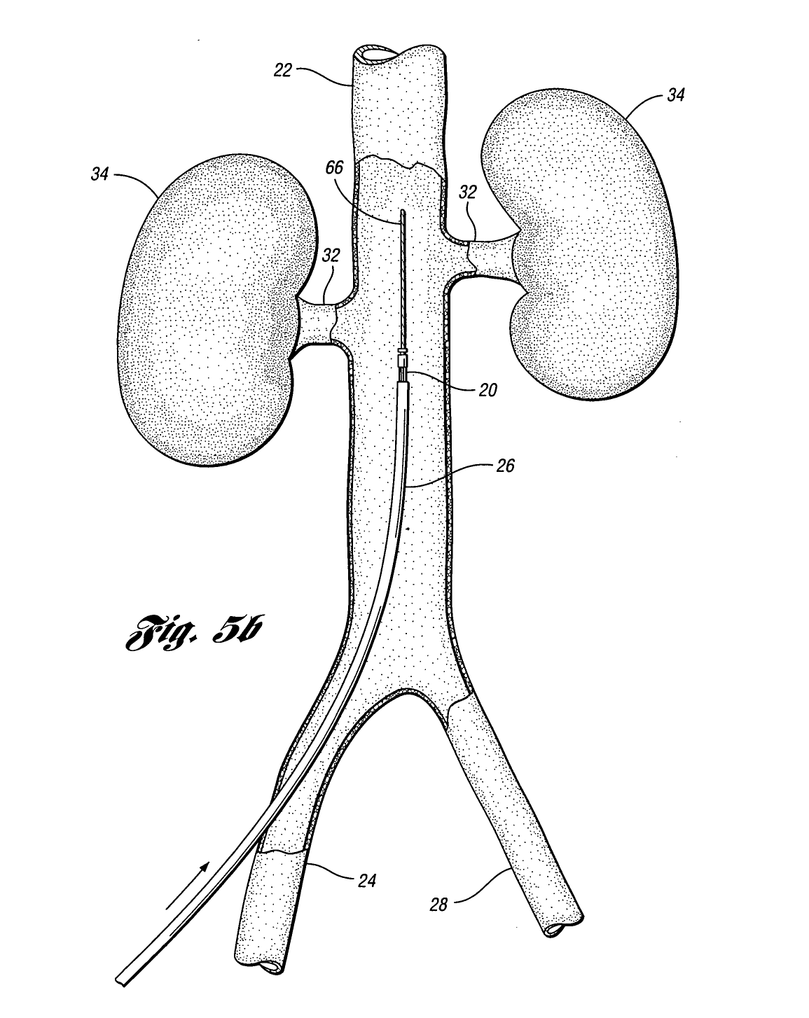

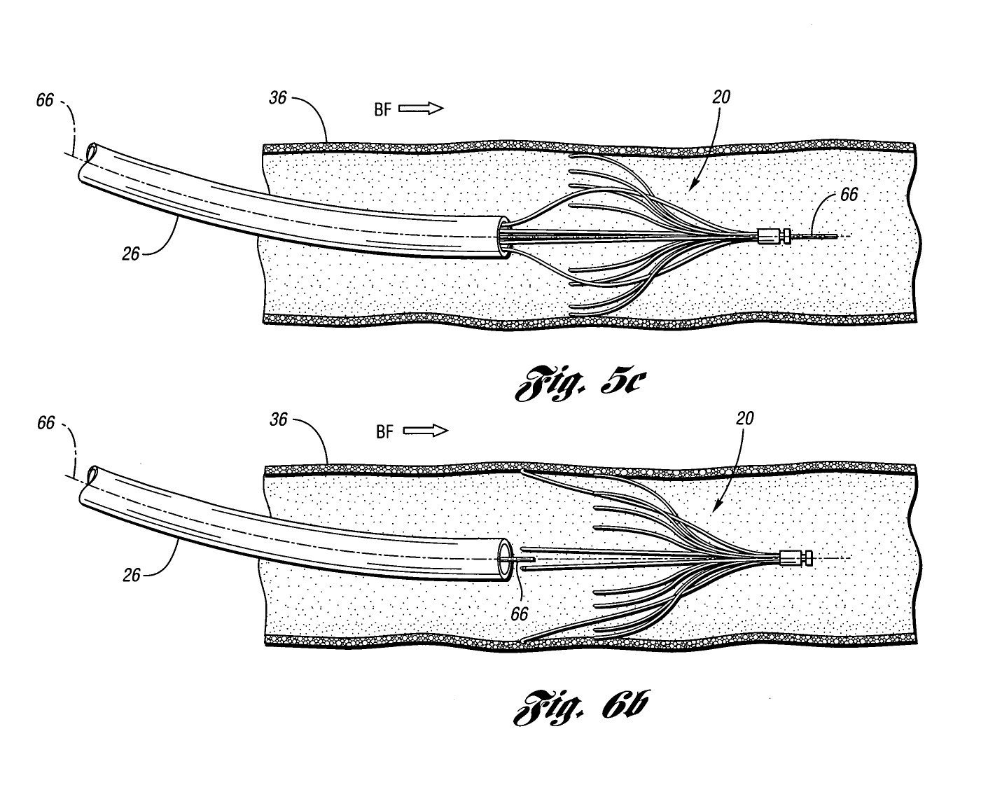

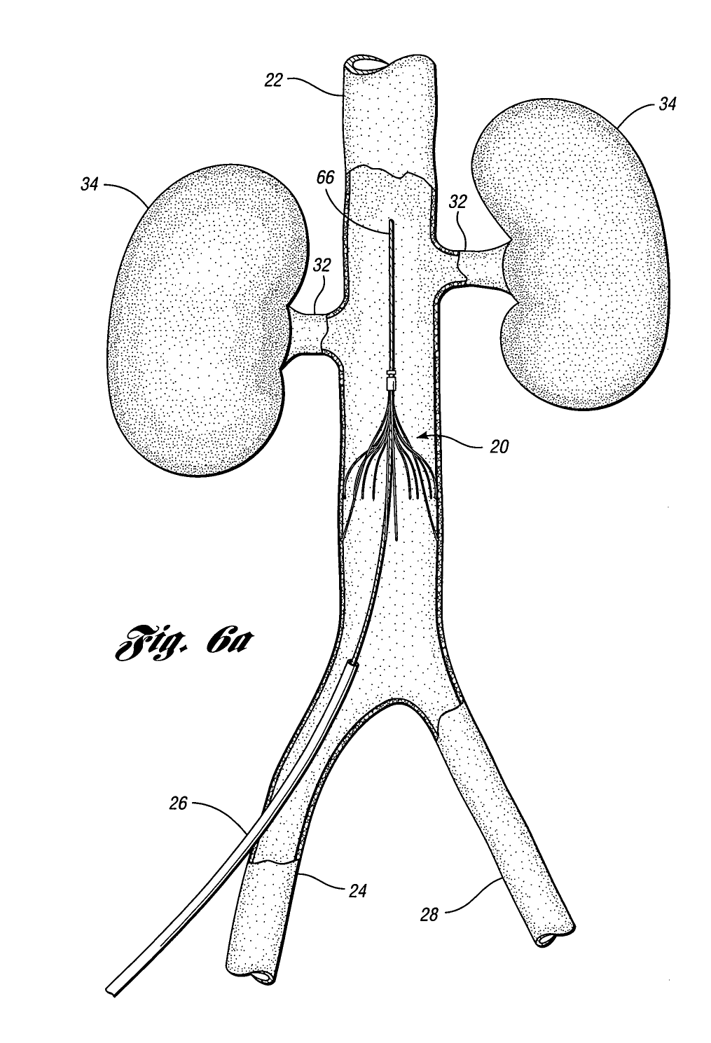

FIGS. 5 and 6 illustrate the deployment of the filter 20 in the vena cava 36, as performed, for example, by a medical specialist such as a physician. Referring in particular to FIG. 5a, the medical specialist insets a wire guide 66 through one of the iliac veins 24 or 28, using, for example, the Seldinger technique, until the distal end of the wire guide 66 is advanced beyond the inferior vena cava 36 to insure seating of the wire guide 66.

Then, as shown in FIG. 5b, the specialist inserts a delivery sheath 26 holding the filter 20 over the wire guide 66 through the puncture site of the patient into the iliac vein 24 and advances the sheath 26 and filter 20 to the deployment site. Note that neither the sheath 26 nor the filter 20 scrape or puncture the inner wall of the blood vessel because they follow the path of the wire guide 66. As such, the sheath 26 is deployed over the wire guide 66 so that the distal end of wire guide 66 extends beyond the distal end of the sheath 26 and the proximal end of the wire guide extends beyond the proximal end of the sheath. Referring to FIG. 5c, the specialist then pushes the filter 20 out of the distal end of the delivery sheath 26 with the free ends of the primary struts 38 held, for example, by a filter retainer member. The filter retainer member may be connected to a pusher member, such as a cannula, that is fed through the proximal end of the delivery sheath 26 until the filter reaches the terminal end of the delivery sheath 26. For a more complete disclosure of the filter delivery system that may be adapted to deliver the filter 20 to a desired location, reference may be made to U.S. Pat. No. 5,324,304 which is incorporated herein by reference in its entirety.

As the filter 20 emerges from the delivery sheath 26, the secondary struts 40 expand to an expanded state to stabilize the attitude of the filter 20 about the center of the blood vessel 36. The specialist pulls the sheath 26 back until the filter 20 is fully deployed in the vena cava 36, as shown in FIG. 6a, and then pulls the wire guide 66 away from the filter, as shown in FIG. 6b, when the specialist is satisfied with the placement of the of the filter 20. The sheath 26 and the wire guide 66 are subsequently removed from the patient.

When fully deployed, the free ends of the primary struts 38 along with the converging section of the secondary struts 40 engage with the vessel wall. The anchoring hooks 52 (FIG. 7) of the primary struts 38 anchor the filter 20 at the location of deployment, preventing the filter 20 from moving with the blood flow (BF) through the vessel. Specifically, as the sheath 26 is pulled back, the barbs 54 are oriented in the direction BF, which along with the outward spring bias of the primary struts 38 causes the anchoring hooks 52 to engage the vessel wall and anchor the filter at the location of deployment. As a result, the filter 20 is supported by the two sets of struts 38, 40 at respective planes of engagement 57, 65 spaced axially along the length of the filter. Moreover, the struts 38, 40 avoid engaging the vessel wall along their lengths to minimize endothelialization in the vessel wall.

With further reference to FIG. 7, the filter 20 is shown fully expanded after being deployed in the inferior vena cava 36. In particular, the anchoring hooks 52 at the ends of the primary struts 38 are shown as being anchored in the inner lining of the inferior vena cava 36. As mentioned above, after deployment of the filter 20, the pressure of the blood flow on the filter 20 contributes in maintaining the barbs 54 anchored in the inner lining of the blood vessel such as the inferior vena cava 36. Also, as noted previously, the converging section 58 of the secondary struts 40 are spring biased to engage with the vessel wall. The engagement of the converging section 58 with the vessel wall functions both initially and after full deployment of the filter to stabilize the attitude of filter 20 about the center of the blood vessel.

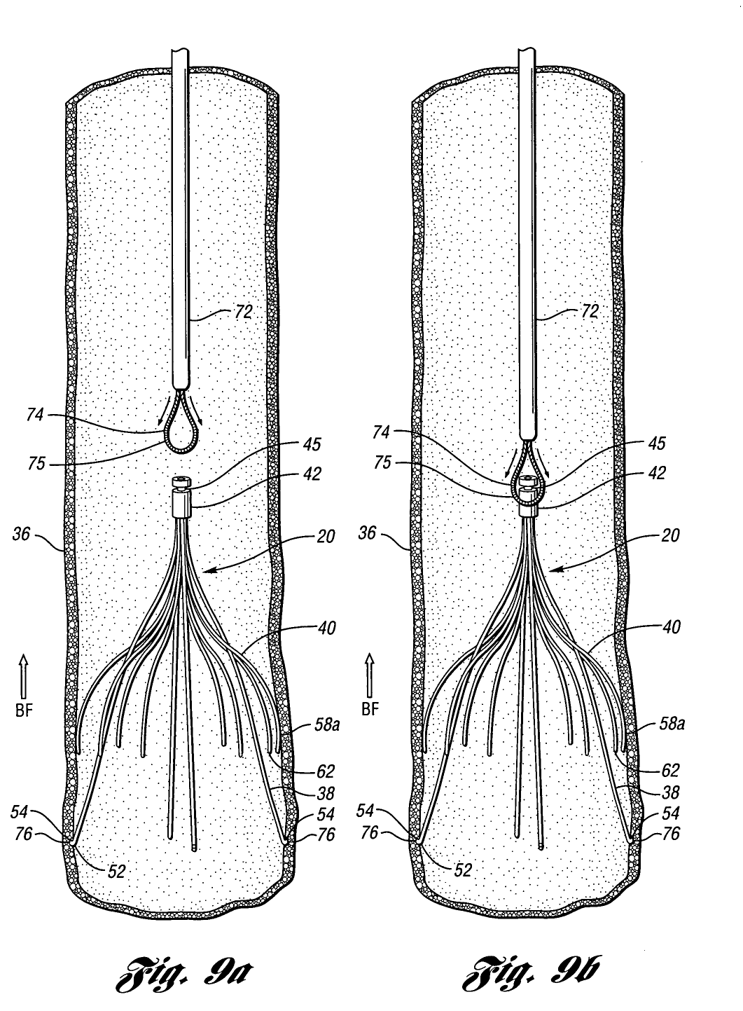

Referring also to FIG. 8 there is shown a netting pattern (“net”) formed by the primary struts 38 and the secondary struts 40 extending from the hub 42. This net catches thrombi carried in the blood stream to prevent the thrombi from reaching the heart and lungs, where the thrombi could cause pulmonary embolism. The size of the net is designed to catch and stop thrombi that are of a size that are undesirable in the vasculature of the patient.

As illustrated in FIG. 8, the struts 38, 40 have substantially equal angular spacing between them. Alternatively, the secondary struts alone may have substantially equal angular spacing between adjacent secondary struts, for example, when the primary struts 38 are employed as the anchoring struts and the secondary struts are employed as the filtering struts. In this alternative implementation, the angle between the primary struts and the adjacent secondary struts is smaller than the angle between adjacent secondary struts.

The filter 20 may be removed percutaneously from the vena cava. To remove the filter 20, the hub 42 is typically grasped about the groove 45 (see FIG. 3) by a retrieval device that is introduced percutaneously in the vena cava.

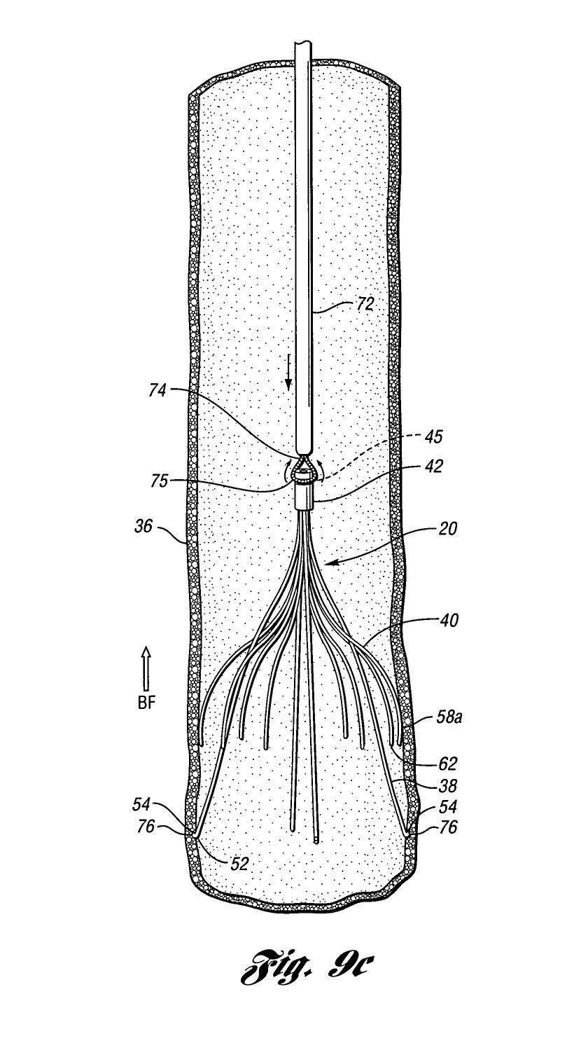

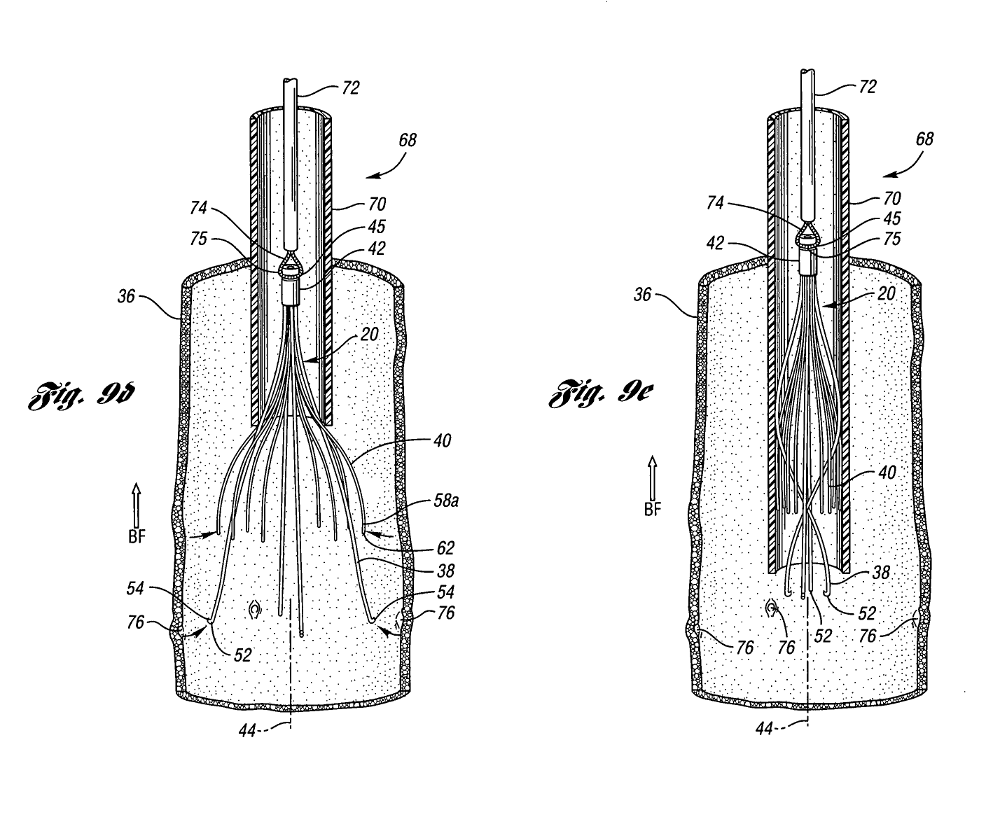

FIGS. 9a through 9e illustrate part of a retrieval device 68 being used, for example, by a medical specialist, for removing the filter 20 from the inferior vena cava 36. The retrieval device 68 includes a removal sheath 70 (FIGS. 9d and 9e) and a snare 74 with a loop 75 inserted through a catheter 72.

Referring to FIG. 9a, the specialist places the catheter 72 into the inferior vena cava 36 and advances the loop portion 75 of the snare 74 out of the distal end of the catheter 72. Then, as shown in FIG. 9b, the specialist positions the loop 75 over the hub 42. The specialist manipulates the snare 74 by any suitable means from the proximal end of the snare 74 such that the loop 75 engages with the groove 45. Once the loop 75 is engaged with the groove 45, the specialist advances the catheter 72 to tighten the loop 75 about the groove 45 as shown in FIG. 9c.

Next, as shown in FIG. 9d, the specialist inserts the sheath 70 into the superior vena cava through the patient's jugular vein and then advances the sheath 70 over the catheter 72. As counter traction is used by pulling the catheter 72 and the snare 74 while pushing the sheath 70, the sheath 70 passes over the filter 20. As the sheath 70 passes over the filter 20, the primary struts 38 and then the secondary struts 40 engage the edge of the end of the sheath 70, causing the struts to pivot at the hub 42 and collapse towards the central axis 44 of the filter 20 (FIG. 9e). This pivoting movement toward the central axis 44 causes the anchoring ends 52 of the primary struts 38 and the converging section 58 of the secondary struts 40 to retract from the inner wall of the vessel 36. In this way, only small point lesions 76 where the anchoring hooks 54 of the primary struts 38 anchored to the vessel wall and surface lesions where the converging section 58 (see FIG. 2) of the secondary struts 48 engaged the vessel wall remain after the removal procedure. It should be noted that removal of the filter 20 from the patient is not limited to the procedure shown in FIG. 9. Other suitable procedures may be employed. For example, the filter 20 may be removed through a femoral vein of the patient.

The foregoing and other implementations of the invention are within the scope of the following claims.

What is claimed is:

1. A filter for capturing blood clots in a blood vessel comprising:
a hub with a passageway through which a wire guide is received;
a plurality of primary struts that extend from the hub and terminate with respective hooks to anchor the filter in the blood vessel when the filter is deployed in the blood vessel; and
a plurality of secondary struts that extend from the hub, the secondary struts centering the filter in the blood vessel as the secondary struts engage the interior of the blood vessel during deployment of the filter in the blood vessel.

2. The filter of claim 1 wherein each of the primary struts and the secondary struts includes a fixed end housed in the hub, the fixed ends of the primary and secondary struts being secured together in a bundle that defines a central axis extending through the passageway, the central axis being substantially parallel to a longitudinal axis extending through the blood vessel when the filter centers itself in the blood vessel.

3. The filter of claim 1 wherein the filter has a collapsed configuration and an expanded configuration, the filter expanding from the collapsed configuration to the expanded configuration as the filter is deployed in the blood vessel, the secondary struts centering the filter as the filter expands to its expanded configuration.

4. The filter of claim 2 wherein the primary struts and secondary struts form a net when the filter is in the expanded configuration to capture blood clots.

5. The filter of claim 1 wherein the hooks are provided with barbs that engage the interior wall of the blood vessel.

6. The filter of claim 1 wherein the primary struts are made of shape memory alloy.

7. The filter of claim 1 wherein the secondary struts are made of shape memory alloy.

8. The filter of claim 1 wherein the primary struts are spaced apart angularly about the passageway, the spacing between the primary struts being substantially equal.

9. The filter of claim 8 wherein a pair of secondary struts is positioned angularly between each pair of spaced apart primary struts.

10. The filter of claim 8 wherein a primary strut is positioned between a respective pair of secondary struts.

11. The filter of claim 1 wherein the plurality of primary struts is four primary struts.

12. The filter of claim 1 wherein the plurality of secondary struts is eight secondary struts.

13. The filter of claim 1 wherein the hub is provided with a groove for retrieving the filter from the blood vessel.

14. A method of deploying a filter in a blood vessel for capturing blood clots comprising:
inserting a wire guide into the blood vessel, the wire guide having a proximal end and a distal end, the proximal end being external to the vessel and the distal end being near the deployment location for the filter;
deploying a sheath over the wire guide, the sheath having a proximal end and a distal end;
inserting the filter into the proximal end of the sheath, the filter including a hub with a passageway through which the wire guide is received, a plurality of primary struts extending from the hub and terminating with respective hooks, and a plurality of secondary struts extending from the hub; and
pushing the filter through the sheath until the filter exits the distal end of the sheath and expands to an expanded configuration, the secondary struts centering the filter in the blood vessel as the secondary struts expand to the expanded configuration and engage the interior of the blood vessel, the primary struts expanding to the expanded configuration upon exiting the distal end of the sheath, and the hooks anchoring the filter in the blood vessel.

15. The method of claim 14 further comprising removing the wire guide from the sheath.

16. The method of claim 14 further comprising removing the sheath from the vessel.

17. The method of claim 14 wherein each of the primary struts and the secondary struts includes a fixed end, the fixed ends of the primary and secondary struts being secured together in a bundle that defines a central axis extending through the passageway, the central axis being substantially parallel to a longitudinal axis extending through the blood vessel when the filter centers itself in the blood vessel.

18. The method of claim 14 wherein the primary and secondary struts form a net when the filter is in the expanded configuration to capture blood clots.

19. The method of claim 14 wherein the hub is provided with a groove for retrieving the filter from the vessel.

20. The method of claim 19 further comprising introducing a retrieval device into the vessel, the retrieval device including a snare that engages with the groove, and pulling the snare and the filter into a retrieval sheath to remove the filter from the vessel.
